(19) United States
(12) Patent Application Publication
(10) Pub. No.: US 2006/0159758 A1
(43) Pub. Date: Jul. 20, 2006

(54) Coating composition for taste masking coating and methods for their application and use

(76) Inventors: Rajesh Gandhi, Madhya Pradesh (IN); Chayapathy Issa, Andhra Pradesh (IN); Rajiv Malik, Vienna (AU)

Correspondence Address: RANBAXY INC., 600 COLLEGE ROAD EAST, SUITE 2100, PRINCETON, NJ, 08540, US

(21) Appl. No.: 10/538,354

(22) PCT Filed: Dec. 11, 2003
(86) PCT No.: PCT/IB03/05877

§ 371 Date: Jan. 3, 2006

(30) Foreign Application Priority Data
Dec. 11, 2002 (IN) 1240/DEL/2002

(51) Int. Cl. A61K 9/24

(52) U.S. Cl. 424/472

(57) ABSTRACT

The present invention relates to coating compositions for taste masking and methods for applying the coating compositions to dosage forms to mask the taste of a medicinal substance. The taste masking coating compositions generally include a copolymer of acrylate and methacrylate with a quaternary ammonium group in combination with sodium carboxymethylcellulose and a polyvinyl alcohol-polyethylene glycol copolymer.

### FIELD OF THE INVENTION

The present invention relates to coating compositions for taste masking and methods for applying the coating compositions to dosage forms to mask the taste of a medicinal substance.

### BACKGROUND OF THE INVENTION

Oral dosage forms are taken by the patient in the form of, for example, solutions, emulsions, suspensions, capsules and tablets. The solid dosage forms having the greatest importance because of their good dosability, packaging, transportability, stability, and ease of administration. As is known in the pharmaceutical arts, many medicinal substances have an unpleasant or bitter taste, which is why either contact of the medicinal substance with the mucosa of the mouth and pharynx is preferentially avoided or the bitter taste is masked. If the dosage form is swallowed whole, the unpleasant taste of the medicinal substance is greatly minimized or avoided altogether. However, children, the elderly, and many other patients have difficulty in swallowing tablets and capsules that have not been broken up. For such patients, pharmaceutically active ingredients are variously formulated as chewable tablets, mouth-dissolving tablets, dispersible tablets, dry powders for reconstitution, or liquid dosage forms. Even with these dosage forms, however, the possibility remains that there will be a perceptible exposure of the active drug to the taste buds; thus, a major requirement of such dosage forms is that they must be palatable. If they are not palatable, the undesirable taste of the formulation creates reluctance in the patient to taking the medicine in that dosage form.

Applying a coating to a dosage form is a known technique for taste masking of bitter medicaments because such coatings provide a barrier that prevents the unpleasant taste of the medicament from coming through, thereby rendering the formulation more palatable. Various types of coatings can be applied to a drug or dosage form. For example, taste masking coatings may employ pH dependent or pH independent polymers.

As another approach, combinations of different polymers may be used to achieve taste masking. Methacrylic acid polymers alone or in combination with other polymers have been used by various researchers to mask the bitter taste of medicaments. When applied alone, increased amounts of polymers are required to mask the bitterness of the medicament being taste masked. Moreover, complete instant release in the entire pH range of the gastrointestinal tract (pH range of between 1 and 8) may not be attained. One of the major drawbacks to the incorporation of methacrylates in increased amounts in formulations relate to perceptions of safety and acceptability of such formulations. It is likely that these perceptions are the reasons why combinations of methacrylate with other polymers have been tried.

For example, U.S. Pat. No. 6,136,347 describes flavor-masked pharmaceutical compositions that include microcapsules. The microcapsules include a coating of water insoluble neutral methacrylic acid ester copolymers and triethylcitrate.

U.S. Pat. No. 6,106,861 describes a rapidly disintegrable multiparticulate tablet which disintegrates in the mouth in less than 40 seconds and includes excipients selected from disintegrating agents, binding agents, and an active ingredient. The active ingredient is in the form of microcrystals coated with a taste masking coating that includes polymethacrylates and cellulose polymers such as hydroxypropyl-methyl cellulose, hydroxypropyl cellulose and cellulose acetophthalates.

PCT application WO 99/44581 describes a process for taste masking of Topiramate by coating the core with a taste masking coating mixture. The taste masking mixture includes cellulose acetate, cellulose acetate butyrate, methylcellulose, ethylcellulose or an Eudragit, and a disintegrant.

PCT application WO 98/14179 describes taste-masked microcapsule formulations for water-soluble drugs in a polymeric material. The polymeric material is described as being one or more polymers selected from ethyl cellulose, cellulose acetate phthalate, cellulose acetate butyrate, polymethacrylates, hydroxypropyl methyl cellulose phthalate, carboxymethyl ethylcellulose, polylactic acid and combinations thereof.

### SUMMARY OF THE INVENTION

In one general aspect there is provided a taste masking coating composition. The taste masking coating composition includes a copolymer of acrylate and methacrylate with a quaternary ammonium group in combination with sodium carboxymethylcellulose and a polyvinyl alcohol-polyethylene glycol copolymer.

Embodiments of the taste masking coating composition may include one or more of the following features. For example, a ratio of the copolymer of acrylate and methacrylate to the copolymer of polyvinyl alcohol-polyethylene glycol may be about 1:2 to 1:3. The concentration of the copolymer of acrylate and methacrylate may be about 20% w/w to about 30% w/w of the taste masking coating composition. The concentration of the copolymer of polyvinyl alcohol-polyethylene glycol may be about 65% w/w to about 75% w/w of the total coating composition.

The taste masking coating composition may further include a lubricant. The lubricant may be one or more of talc, glyceryl monostearate, magnesium stearate, and colloidal silica. The lubricant may be up to 10% of the dry weight of the taste masking coating composition.

The taste masking coating composition may be coated on one or more of a core, granule, pellet, active pharmaceutical ingredient, or dosage form, the core, granule, pellet, or dosage form containing an active pharmaceutical ingredient.

The taste masking coating composition may release more than 60% of the active pharmaceutical ingredient in 15 minutes, more than 80% of the active pharmaceutical ingredient in 30 minutes, and more than 90% of the active pharmaceutical ingredient in 45 minutes when the core, granule, pellet, or dosage form is placed in 900 ml of a glycine buffer (pH 3.0) with apparatus 2 with stirring at 75 RPM and aliquots of the solution are analyzed spectrophotometrically at a wavelength of 259 nm.

In another general aspect there is provided an immediate release, taste-masked pharmaceutical composition for oral administration. The pharmaceutical composition includes a core, an active pharmaceutical ingredient, and a taste masking coating. The core includes the active pharmaceutical ingredient. The taste masking coating may include a combination of (i) copolymers of acrylate and methacrylate with a quaternary ammonium group in combination with sodium carboxymethylcellulose and (ii) polyvinyl alcohol-polyethylene glycol copolymer. The core and the active pharmaceutical ingredient are coated with the taste masking coating.

Embodiments of the pharmaceutical composition may include one or more of the following features. For example, the pharmaceutical composition may release more than 60% of the active pharmaceutical ingredient in 15 minutes, more than 80% of the active pharmaceutical ingredient in 30 minutes, and more than 90% of the active pharmaceutical ingredient in 45 minutes when the taste mask coated core is placed in 900 ml of a glycine buffer (pH 3.0) with apparatus 2 with stirring at 75 RPM and aliquots of the solution are analyzed spectrophotometrically at a wavelength of 259 nm.

The ratio of (i) to (ii) may be about 1:2 to about 1:3. The concentration of (i) may be between about 20% w/w and about 30% w/w of the taste masking coating. The concentration of (ii) may be between about 65% w/w and about 75% w/w of the total coating composition.

The taste masking coating may further include one or more lubricants. The lubricant may be one or more of talc, glyceryl monostearate, magnesium stearate, and colloidal silica. The lubricant may be up to 10% of the dry weight of the taste masking coating composition. The taste masking coating may be between about 10% w/w and about 40% w/w of the core and active pharmaceutical ingredient and, more particularly, may be between about 10% w/w and about 25% w/w of the core and active pharmaceutical ingredient.

The core may be one or more of an insoluble material, a soluble material, and a swellable material. The core may be an insoluble material and the insoluble material may be one or more of sand, glass, microcrystalline cellulose, and plastic. The core may be a soluble material and the soluble material may be one or more sugars including glucose, mannitol, lactose, xylitol, dextrose, sucrose, and mixtures thereof. The core may be a swellable material such as hydroxypropyl methylcellulose.

The active pharmaceutical ingredient may be one or more of alkaloids, antacids, analgesics, anabolic agents, anti-anginal drugs, anti-allergy agents, anti-arrhythmia agents, antiasthmatics, antibiotics, anticholesterolemics, anticonvulsants, anticoagulants, antidepressants, antidiarrheal preparations, anti-emetics, antihistamines, antihypertensives, anti-infectives, anti-inflammatories, antilipid agents, antimanics, anti-migraine agents, antinauseants, antipsychotics, antistroke agents, antithyroid preparations, anabolic drugs, antiobesity agents, antiparasitics, antipsychotics, antipyretics, antispasmodics, antithrombotics, antitumor agents, antitussives, antiulcer agents, anti-uricemic agents, anxiolytic agents, appetite stimulants, appetite suppressants, beta-blocking agents, bronchodilators, cardiovascular agents, cerebral dilators, chelating agents, cholecystekinin antagonists, chemotherapeutic agents, cholesterol reducing agents, cognition activators, contraceptives, coronary dilators, cough suppressants, CNS drugs, decongestants, diabetes agents, diuretics, emollients, enzymes, erythropoietic drugs, expectorants, fertility agents, fungicides, gastrointestinal agents, growth regulators, hormone replacement agents, hyperglycemic agents, hypoglycemic agents, ion-exchange resins, laxatives, migraine treatments, mineral supplements, mucolytics, narcotics, neuroleptics, neuromuscular drugs, non-steroidal anti-inflammatories (NSAIDs), nutritional additives, peripheral vasodilators, polypeptides, prostaglandins, psychotropics, renin inhibitors, respiratory stimulants, sedatives, steroids, stimulants, sympatholytics, thyroid preparations, tranquilizers, uterine relaxants, vaginal preparations, vasoconstrictors, vasodilators, vertigo agents, vitamins, and wound healing agents.

The analgesic may be one or more of acetaminophen, aspirin, ibuprofen, naproxen, and ketoprofen. The antibiotic may be one or more of cefuroxime axetil, cefpodoxime proxetil, ciprofloxacin, erythromycin, and clarithromycin and, in particular, may be cefpodoxime proxetil. The gastrointestinal agent may be one or more of loperamide, famotidine, ranitidine, and cimetidine. The cardiovascular agents may be one or more of irbesartan, captopril, and lisinopril. The CNS drug may be one or more of nefazodone and buspirone. The antihistamine may be one or more of chlorpheniramine and astemizole. The cholesterol reducing agent may be a statin, e.g., atorvastatin, simvastatin, pravastatin, and lovastatin.

The taste-masked pharmaceutical composition may be in the form of one or more of sprinkles, dry powder, suspension, emulsion, whole chewable tablets, and dispersible tablets.

The taste-masking coating may be applied to the active pharmaceutical ingredient. The taste masking coating may further include one or more of plasticizers, coloring agents, and gloss producers.

In another general aspect there is provided a process for preparing a taste masking coating composition. The process includes combining (i) a copolymer of acrylate and methacrylate with a quaternary ammonium group in combination with sodium carboxymethylcellulose and (ii) a polyvinyl alcohol-polyethylene glycol copolymer.

Embodiments of the process may include one or more of the following features. For example, the process may further include adding one or more of a lubricant, a plasticizer, a coloring agent, and a gloss producer.

In another general aspect there is provided a process for preparing an immediate release taste-masked pharmaceutical composition for oral administration. The process includes coating a core containing an active pharmaceutical ingredient with a taste masking coating composition. The taste masking coating composition includes a combination of (i) copolymers of acrylate and methacrylate with a quaternary ammonium group in combination with sodium carboxymethylcellulose and (ii) a polyvinyl alcohol-polyethylene glycol copolymer.

Embodiments of the process may include one or more of the following features. For example, the ratio of (i) to (ii) may be between about 1:2 and about 1:3. The concentration of (i) may be between about 20% w/w and about 30% w/w of the total coating composition. The concentration of (ii) may be between about 65% w/w and about 75% w/w of the total coating composition.

The taste masking coating composition may further include one or more lubricants. The lubricant may be one or more of talc, glyceryl monostearate, magnesium stearate, and colloidal silica. The lubricant may be up to about 10% of the dry weight of the taste masking coating composition.

The coating may be between about 10% w/w and about 40% w/w of the active pharmaceutical ingredient-containing core and, more particularly, between about 10% w/w and about 25% w/w of the active pharmaceutical ingredient-containing core.

The core may be one or more of an insoluble material, a soluble material, and a swellable material. The core may be an insoluble material and the insoluble material may be one or more of sand, glass, microcrystalline cellulose, and plastic. The core may be a soluble material and the soluble material may be one or more sugars including glucose, mannitol, lactose, xylitol, dextrose, sucrose, and mixtures thereof. The core may be a swellable material such as hydroxypropyl methylcellulose.

The active pharmaceutical ingredient may be one or more of alkaloids, antacids, analgesics, anabolic agents, anti-anginal drugs, anti-allergy agents, anti-arrhythmia agents, antiasthmatics, antibiotics, anticholesterolemics, anticonvulsants, anticoagulants, antidepressants, antidiarrheal preparations, anti-emetics, antihistamines, antihypertensives, anti-infectives, anti-inflammatories, antilipid agents, antimanics, anti-migraine agents, antinauseants, antipsychotics, antistroke agents, antithyroid preparations, anabolic drugs, antiobesity agents, antiparasitics, antipsychotics, antipyretics, antispasmodics, antithrombotics, antitumor agents, antitussives, antiulcer agents, anti-uricemic agents, anxiolytic agents, appetite stimulants, appetite suppressants, beta-blocking agents, bronchodilators, cardiovascular agents, cerebral dilators, chelating agents, cholecystekinin antagonists, chemotherapeutic agents, cholesterol reducing agents, cognition activators, contraceptives, coronary dilators, cough suppressants, CNS drugs, decongestants, diabetes agents, diuretics, emollients, enzymes, erythropoietic drugs, expectorants, fertility agents, fungicides, gastrointestinal agents, growth regulators, hormone replacement agents, hyperglycemic agents, hypoglycemic agents, ion-exchange resins, laxatives, migraine treatments, mineral supplements, mucolytics, narcotics, neuroleptics, neuromuscular drugs, non-steroidal anti-inflammatories (NSAIDs), nutritional additives, peripheral vasodilators, polypeptides, prostaglandins, psychotropics, renin inhibitors, respiratory stimulants, sedatives, steroids, stimulants, sympatholytics, thyroid preparations, tranquilizers, uterine relaxants, vaginal preparations, vasoconstrictors, vasodilators, vertigo agents, vitamins, and wound healing agents.

The analgesic may be one or more of acetaminophen, aspirin, ibuprofen, naproxen, and ketoprofen. The antibiotic may be one or more of cefuroxime axetil, cefpodoxime proxetil, ciprofloxacin, erythromycin, and clarithromycin and, in particular, may be cefpodoxime proxetil. The gastrointestinal agent may be one or more of loperamide, famotidine, ranitidine, and cimetidine. The cardiovascular agents may be one or more of irbesartan, captopril, and lisinopril. The CNS drug may be one or more of nefazodone and buspirone. The antihistamine may be one or more of chlorpheniramine and astemizole. The cholesterol reducing agent may be a statin, e.g., atorvastatin, simvastatin, pravastatin, and lovastatin.

The process may further include formulating the taste-masked pharmaceutical composition as sprinkles, a dry powder, a suspension, an emulsion, whole chewable tablets, or dispersible tablets.

The taste-masking coating composition may be applied to the drug. The taste masking coating composition may further include one or more of a plasticizer, a coloring agent, and a gloss producer.

In another general aspect there is provided a process for preparing a taste-masked pharmaceutical composition. The process includes coating one or more microcrystalline cellulose beads with a suspension containing at least one active pharmaceutical ingredient to form one or more drug loaded beads and coating the drug loaded bead with a taste masking coating composition. The taste masking coating composition includes (i) 25% w/w of the total taste masking coating composition of a copolymer of acrylate and methacrylate with a quaternary ammonium group in combination with sodium carboxymethylcellulose and (ii) 68.5% w/w of the total taste masking coating composition of polyvinyl alcohol-polyethylene glycol copolymer. Embodiments of the process may include any one of the features described above.

In another general aspect there is provided a method of treating, preventing or diagnosing a disease condition by orally administering a taste-masked pharmaceutical composition to a patient in need thereof. The pharmaceutical composition includes a core containing an active pharmaceutical ingredient and a taste masking coating composition. The taste masking coating composition forms a coat around at least a portion of the core and includes a combination of (i) a copolymer of acrylate and methacrylate with a quaternary ammonium group in combination with sodium carboxymethylcellulose and (ii) a polyvinyl alcohol-polyethylene glycol copolymer.

The details of one or more embodiments of the inventions are set forth in the description below. Other features, objects and advantages of the inventions will be apparent from the description and claims.

### DETAILED DESCRIPTION OF THE INVENTION

As described above, a number of taste masking coating compositions are known. None of these compositions, however, are fully satisfactory as complete taste masking combined with rapid release cannot be achieved using these compositions. Therefore, the inventors believed there to be a need for a taste masking composition that can provide a dosage form that is both palatable and bioavailable. The inventors have satisfied the above needs by using coating compositions that includes a combination of (i) copolymers of acrylate and methacrylate with a quaternary ammonium group in combination with sodium carboxymethylcellulose and (ii) polyvinyl alcohol-polyethylene glycol copolymer.

Surprisingly, the inventors have found that when combinations of these two polymers are used as taste masking coating compositions, the release rate of the medicament is increased and optimal results are observed with respect to taste masking and release of active components. Moreover, the amount of acrylate and methacrylate copolymers with a quaternary ammonium group in combination with sodium carboxymethylcellulose required for coating can also be reduced, thereby, ensuring the safety and acceptability of the dosage form.

Copolymers of acrylate and methacrylate with a quaternary ammonium group in combination with sodium carboxymethylcellulose is available under the trade name Eudragit RD 100 supplied by Rohm GmbH, Darmstadt. This copolymer provides pH independent, fast disintegrating films and coatings that are especially suitable for taste masking purposes. A disintegrant, sodium carboxymethylcellulose, is inherently present in the Eudragit RD 100 and thereby facilitates the fast release of the medicament.

Polyvinyl alcohol-polyethylene glycol copolymers are commercially available under the trade name Kollicoat IR and are marketed by BASF Corporation. This copolymer is highly soluble in water and is used as a covering or coating for instantaneous release in tablets.

The inventors have found that the combination of these copolymers can be used to formulate an immediate release taste-masked pharmaceutical composition for oral administration. In such formulations, a core containing the bitter or unpleasant tasting drug is coated with a combination of (i) copolymers of acrylate and methacrylate with a quaternary ammonium group in combination with sodium carboxymethylcellulose and (ii) polyvinyl alcohol-polyethylene glycol copolymer.

The term “immediate release” as used herein means release of the medicament in the gastrointestinal tract within approximately one hour.

As described in further detail below, the combination of the copolymers can be prepared as a general taste masking coating that can be applied to almost any medicament to mask the bitter or undesirable taste of the medicament without also delaying the availability of the medicament when consumed orally.

Further, a pharmaceutical composition using the combination of copolymers can be used in a method of treating, preventing or diagnosing a disease condition that includes orally administering a taste-masked pharmaceutical composition. As described in further detail herein, the pharmaceutical composition includes a core containing the bitter or otherwise unpleasant tasting drug. This drug containing or drug loaded core is coated with a combination of (i) copolymers of acrylate and methacrylate with a quaternary ammonium group in combination with sodium carboxymethylcellulose and (ii) polyvinyl alcohol-polyethylene glycol copolymer. Because of the taste masking, the pharmaceutical composition can be orally administered without the concern that the composition will be unpalatable.

The drug-containing core may be selected from one or more of pharmaceutically inert insoluble materials, soluble material, and swellable materials. The insoluble inert cores may be, for example, sand (i.e., silicon dioxide), glass, microcrystalline cellulose (e.g., celpheres) or a plastic material (e.g., polystyrene). The soluble inert cores may be a sugar selected from one or more of glucose, mannitol, lactose, xylitol, dextrose, sucrose and the like. The swellable inert cores may be, for example, hydroxypropyl methylcellulose or any other suitable swellable inert material. As described below, the drug is loaded on the core by coating or spraying of the taste masking coating composition.

In addition to the above two copolymers, the coating composition also may contain lubricants that function as anti-sticking agents. These lubricants may be selected from talc, glyceryl monostearate, magnesium stearate, colloidal silica, other suitable lubricants, and mixtures thereof. The concentration of lubricant in the composition may be up to 10% of the dry weight of the taste masking coating composition.

The taste masking coating composition can be prepared in numerous ways. For example, the polyvinyl alcohol-polyethylene glycol copolymer may be dispersed in purified water under stirring to form a solution. Eudragit then is dispersed in the solution under constant stirring. Talc next is added and the stirring is continued for approximately twenty minutes. Following this stirring, the coating suspension is filtered through a 250 micron nylon cloth. This coating composition then can be applied to taste mask bitter medicaments by using any suitable procedure, such as spray coating, pan coating, fluidized bed coating, etc.

In the coating procedure, the bitter, unpleasant tasting active ingredient can be directly coated with the coating composition. Alternatively, a drug loaded core can be coated with the taste masking coating suspension in a fluid bed processor to obtain the desired taste masked product.

As described above, the taste masking coating may be a combination of (i) copolymers of acrylate and methacrylate with a quaternary ammonium group in combination with sodium carboxymethylcellulose and (ii) polyvinyl alcohol-polyethylene glycol copolymer.

The copolymer of acrylate and methacrylate with a quaternary ammonium group in combination with sodium carboxymethylcellulose and polyvinyl alcohol-polyethylene glycol copolymer may be present in a ratio of about 1:2 to about 1:3, although formulations that are either below or above this range also are contemplated.

The concentration of methacrylate-acrylate copolymer may be used at about 20% w/w to about 30% w/w and polyvinyl alcohol-polyethylene glycol copolymer at about 65% w/w to about 75% w/w of the total taste masking coating composition.

The coating composition may be used to mask the taste of any category of bitter drugs. For example, the drug can be selected from alkaloids, antacids, analgesics, anabolic agents, anti-anginal drugs, anti-allergy agents, anti-arrhythmia agents, antiasthmatics, antibiotics, anticholesterolemics, anticonvulsants, anticoagulants, antidepressants, antidiarrheal preparations, anti-emetics, antihistamines, antihypertensives, anti-infectives, anti-inflammatories, antilipid agents, antimanics, anti-migraine agents, antinauseants, antipsychotics, antistroke agents, antithyroid preparations, anabolic drugs, antiobesity agents, antiparasitics, antipsychotics, antipyretics, antispasmodics, antithrombotics, antitumor agents, antitussives, antiulcer agents, anti-uricemic agents, anxiolytic agents, appetite stimulants, appetite suppressants, beta-blocking agents, bronchodilators, cardiovascular agents, cerebral dilators, chelating agents, cholecystekinin antagonists, chemotherapeutic agents, cholesterol reducing agents, cognition activators, contraceptives, coronary dilators, cough suppressants, CNS drugs, decongestants, diabetes agents, diuretics, emollients, enzymes, erythropoietic drugs, expectorants, fertility agents, fungicides, gastrointestinal agents, growth regulators, hormone replacement agents, hyperglycemic agents, hypoglycemic agents, ion-exchange resins, laxatives, migraine treatments, mineral supplements, mucolytics, narcotics, neuroleptics, neuromuscular drugs, non-steroidal anti-inflammatories (NSAIDs), nutritional additives, peripheral vasodilators, polypeptides, prostaglandins, psychotropics, renin inhibitors, respiratory stimulants, sedatives, steroids, stimulants, sympatholytics, thyroid preparations, tranquilizers, uterine relaxants, vaginal preparations, vasoconstrictors, vasodilators, vertigo agents, vitamins, wound healing agents, and others.

The analgesics may be such specific drugs as acetaminophen, aspirin, ibuprofen, naproxen, and ketoprofen. The antibiotics may be such specific drugs as cefuroxime axetil, cefpodoxime proxetil, ciprofloxacin, erythromycin, and clarithromycin. The gastrointestinal drugs may be such drugs as loperamide, famotidine, ranitidine, cimetidine and salts thereof. The cardiovascular agents may be such drugs as irbesartan, captopril, lisinopril and salts thereof. The CNS drugs may be such drugs as nefazodone, buspirone and salts thereof. The antihistamines may be such drugs as chlorpheniramine and astemizole. The cholesterol reducing agents may be such drugs as statins, e.g., atorvastatin, simvastatin, pravastatin, and lovastatin. All of these general classes of drugs and the specific drugs are expected to be capable of taste masking using the coating composition described herein.

The coated core can be formulated as sprinkles, dry powder, suspension, emulsion, or as whole a chewable or dispersible tablet, or any other suitable oral dosage forms, including conventional tables and capsules.

Coating additives may be selected from one or more of plasticizers, coloring agents and gloss producers. The plasticizer may be selected from one or more of diethyl phthalate, dibutyl phthalate, triethyl citrate and polyethylene glycol.

The coating composition also can be applied to a whole dosage form and thereby conceal the bitter taste of the medicament contained within.

The following examples are provided merely to illustrate embodiments of the invention and are not intended to limit the scope of the invention.

### EXAMPLE 1

### Dry Suspension of Cefpodoxime Proxetil

Drug Layer Ingredients

| | | | |
|---|---|---|---|
| 1. | Microcrystalline cellulose beads | 190.0 | mg |
| 2. | Cefpodoxime Proxetil (Equivalent to 100 mg | 142.4 | mg |
| | cefpodoxime) | | |
| 3. | Hydroxypropyl methylcellulose | 40.0 | mg |
| 4. | Hydroxy propyl cellulose | 20.0 | mg |
| 5. | Croscarmellose sodium | 15.6 | mg |

| | | |
|---|---|---|
| 6. | Purified water | qs |
| 7. | Isopropyl alcohol | qs |

Taste Masking Layer Ingredients

| | | |
|---|---|---|
| 1. Drug loaded beads | 410.0 | mg |
| 2. Eudragit RD 100 | 25.0 | mg |
| 3. Kollicoat IR | 68.5 | mg |
| 4. Talc | 6.5 | mg |

| | |
|---|---|
| 5. Water | qs |

Composition of the Dry Suspension

| | | | |
|---|---|---|---|
| 1. | Drug coated, taste mask coated beads | 510.0 | mg |
| 2. | Fruit Gum flavor | 15.0 | mg |
| 3. | Frescofort flavor | 15.0 | mg |
| 4. | Colloidal silicon dioxide | 17.5 | mg |
| 5. | Carrageenan | 30.0 | mg |
| 6. | Microcrystalline cellulose | 10.0 | mg |
| 7. | Sodium citrate | 5.0 | mg |
| 8. | Citric acid (Anhydrous) | 3.0 | mg |
| 9. | Ferric oxide (Yellow) | 0.05 | mg |
| 10. | Sucrose | 2994.45 | mg |

Procedure

A liquid suspension of cefpodoxime proxetil and the combination of binders in water was prepared. Frothing was minimized using a small volume of isopropyl alcohol. The liquid suspension was sprayed onto the microcrystalline cellulose beads (MCC beads) and dried to provide core beads using a fluid bed processor. The core beads then were screened to remove fines and agglomerates. The core beads were coated again with a taste masking coating (Eudragit RD 100, Kollicoat IR, Talc, and Water) and dried in a fluid bed processor. The coated beads were sifted to remove fines and agglomerates. The coated beads were mixed with the various remaining ingredients to form the composition of the dry suspension. The final composition was optionally encapsulated.

The in-vitro dissolution release profile of the cefpodoxime proxetil from the dry suspension of Example 1 was determined in accordance with the procedure described in Pharmacopoeial Forum, Vol. 23, Number 4, July-August 1997, pages 4388-4392. In the procedure a weight equivalent to 5 ml suspension was added to 900 ml of glycine buffer (pH 3.0) to form a solution. In this procedure, apparatus 2 with stirring at 75 RPM is used. Aliquots of 5 ml of the solution were taken at 15, 30 and 45 minutes and analyzed spectrophotometrically at a wavelength of 259 nm. The results of the dissolution testing are provided below in Table 1. As can be seen in Table 1, greater than 60% of the drug is released in 15 minutes, greater than 80% of the drug is released in 30 minutes, and greater than 90% of the drug is released in 45 minutes.

TABLE 1
In-vitro dissolution release of the dry suspension of Example 1

| Time (in minutes) | % Drug released |
|---|---|
| 15 | 62.2 |
| 30 | 85.6 |
| 45 | 95.1 |

### EXAMPLE 2

### Immediate Release Pellet Composition of Cefpodoxime Proxetil

Drug Layering

| | | | |
|---|---|---|---|
| 1. | Microcrystalline cellulose beads | 190.0 | mg |
| 2. | Cefpodoxime Proxetil (Equivalent to 100 mg | 142.4 | mg |
| | cefpodoxime) | | |
| 3. | Hydroxypropyl methylcellulose | 40.0 | mg |
| 4. | Hydroxy propyl cellulose | 20.0 | mg |
| 5. | Croscarmellose sodium | 15.6 | mg |

| | | |
|---|---|---|
| 6. | Purified water | qs |
| 7 | Isopropyl alcohol | qs |

Taste Masking Layer Ingredients

| | | |
|---|---|---|
| 1. Drug loaded beads | 410.0 | mg |
| 2. Eudragit RD 100 | 25.0 | mg |
| 3. Kollicoat IR | 68.5 | mg |
| 4. Talc | 6.5 | mg |

| | |
|---|---|
| 5. Water | qs |

Hydroxypropyl methylcellulose, hydroxypropyl cellulose and croscarmellose sodium were dispersed in purified water under stirring. Cefpodoxime proxetil then was dispersed in the above mixture under constant stirring. Isopropyl alcohol was added and stirring was continued for thirty minutes. Next, microcrystalline cellulose beads were coated with this cefpodoxime proxetil dispersion in a fluid bed processor to form granules. The granules were dried until a limit of detection (LOD) of NMT 4.0% at 105° C. (on IR Balance). The dried pellets were coated with the taste masking coating suspension in a fluid bed processor to achieve pellets of the desired product.

The in-vitro dissolution release of drug from the pellets of Example 2 was determined in accordance with the procedure described in Pharmacopoeial Forum, Vol. 23, Number 4, July-August 1997, pages 4388-4392. A 0.510 gm sample of the coated pellets was added to 900 ml of glycine buffer (pH 3.0) to form a solution. In this procedure, apparatus 2 with stirring at 75 RPM is used. Aliquots of 5 ml of the solution were taken at 15, 30 and 45 minutes and analyzed spectrophotometrically at a wavelength of 259 nm. The results of the dissolution testing are provided below in Table 2. As can be seen in Table 2, greater than 70% of the drug is released in 15 minutes, greater than 85% of the drug is released in 30 minutes, and greater than 95% of the drug is released in 45 minutes.

TABLE 2
In-vitro dissolution profile of pellets of Example 2

| Time (in minutes) | % Drug released |
|---|---|
| 15 | 75.1 |
| 30 | 90.3 |
| 45 | 97.8 |

While several particular forms of the inventions have been described, it will be apparent that various modifications and combinations of the inventions detailed in the text can be made without departing from the spirit and scope of the inventions. Further, it is contemplated that any single feature or any combination of optional features of the inventive variations described herein may be specifically excluded from the claimed inventions and be so described as a negative limitation. Accordingly, it is not intended that the inventions be limited, except as by the appended claims.

1. (canceled)

2. (canceled)

3. (canceled)

4. (canceled)

5. (canceled)

6. (canceled)

7. (canceled)

8. (canceled)

9. (canceled)

10. An immediate release, taste-masked pharmaceutical composition for oral administration, the pharmaceutical composition comprising
a core;
an active pharmaceutical ingredient, wherein the core includes the active pharmaceutical ingredient; and
a taste masking coating, the taste masking coating comprising a combination of (i) copolymers of acrylate and methacrylate with a quaternary ammonium group in combination with sodium carboxymethylcellulose and (ii) polyvinyl alcohol-polyethylene glycol copolymer,
wherein the core and the active pharmaceutical ingredient are coated with the taste masking coating.

11. The pharmaceutical composition of claim 10, wherein more than 60% of the active pharmaceutical ingredient is released in about 15 minutes, more than 80% of the active pharmaceutical ingredient is released in about 30 minutes, and more than 90% of the active pharmaceutical ingredient is released in about 45 minutes when the pharmaceutical composition is placed in 900 ml of a glycine buffer (pH 3.0) with apparatus 2 with stirring at 75 RPM and aliquots of the solution are analyzed spectrophotometrically at a wavelength of 259 nm.

12. The pharmaceutical composition of claim 10, wherein the ratio of (i) and (ii) is about 1:2 to about 1:3.

13. The pharmaceutical composition of claim 10, wherein the concentration of (i) is between about 20% w/w and about 30% w/w of the taste masking coating.

14. The pharmaceutical composition of claim 10, wherein the concentration of (ii) is between about 65% w/w and about 75% w/w of the total coating composition.

15. The pharmaceutical composition of claim 10, wherein the taste masking coating further comprises one or more lubricants.

16. (canceled)

17. The pharmaceutical composition of claim 15, wherein the lubricant comprises up to 10% of the dry weight of the taste masking coating composition.

18. The pharmaceutical composition of claim 10, wherein the taste masking coating comprises between about 10% w/w and about 40% w/w of the core and active pharmaceutical ingredient.

19. (canceled)

20. (canceled)

21. (canceled)

22. (canceled)

23. (canceled)

24. (canceled)

25. (canceled)

26. (canceled)

27. The pharmaceutical composition of claim 10, wherein the active pharmaceutical ingredient comprises one or more of alkaloids, antacids, analgesics, anabolic agents, anti-anginal drugs, anti-allergy agents, anti-arrhythmia agents, antiasthmatics, antibiotics, anticholesterolemics, anticonvulsants, anticoagulants, antidepressants, antidiarrheal preparations, anti-emetics, antihistamines, antihypertensives, anti-infectives, anti-inflammatories, antilipid agents, antimanics, anti-migraine agents, antinauseants, antipsychotics, antistroke agents, antithyroid preparations, anabolic drugs, antiobesity agents, antiparasitics, antipsychotics, antipyretics, antispasmodics, antithrombotics, antitumor agents, antitussives, antiulcer agents, anti-uricemic agents, anxiolytic agents, appetite stimulants, appetite suppressants, beta-blocking agents, bronchodilators, cardiovascular agents, cerebral dilators, chelating agents, cholecystekinin antagonists, chemotherapeutic agents, cholesterol reducing agents, cognition activators, contraceptives, coronary dilators, cough suppressants, CNS drugs, decongestants, diabetes agents, diuretics, emollients, enzymes, erythropoietic drugs, expectorants, fertility agents, fungicides, gastrointestinal agents, growth regulators, hormone replacement agents, hyperglycemic agents, hypoglycemic agents, ion-exchange resins, laxatives, migraine treatments, mineral supplements, mucolytics, narcotics, neuroleptics, neuromuscular drugs, non-steroidal anti-inflammatories (NSAIDs), nutritional additives, peripheral vasodilators, polypeptides, prostaglandins, psychotropics, renin inhibitors, respiratory stimulants, sedatives, steroids, stimulants, sympatholytics, thyroid preparations, tranquilizers, uterine relaxants, vaginal preparations, vasoconstrictors, vasodilators, vertigo agents, vitamins, and wound healing agents.

28. (canceled)

29. (canceled)

30. (canceled)

31. (canceled)

32. (canceled)

33. (canceled)

34. (canceled)

35. (canceled)

36. (canceled)

37. The pharmaceutical composition of claim 10, wherein the taste-masking coating is applied to the active pharmaceutical ingredient.

38. The pharmaceutical composition of claim 10, wherein the taste masking coating further comprises one or more of plasticizers, coloring agents, and gloss producers.

39. (canceled)

40. (canceled)

41. A process for preparing an immediate release taste-masked pharmaceutical composition for oral administration, the process comprising:
coating a core containing an active pharmaceutical ingredient with a taste masking coating composition, the taste masking coating composition comprising a combination of (i) copolymers of acrylate and methacrylate with a quaternary ammonium group in combination with sodium carboxymethylcellulose and (ii) a polyvinyl alcohol-polyethylene glycol copolymer.

42. The process of claim 41, wherein the ratio of (i) to (ii) is between about 1:2 and about 1:3.

43. The process of claim 41, wherein the concentration of (i) is between about 20% w/w and about 30% w/w of the total coating composition.

44. The process of claim 41, wherein the concentration of (ii) is between about 65% w/w and about 75% w/w of the total coating composition.

45. The process of claim 41, wherein the taste masking coating composition further comprises one or more lubricants.

46. (canceled)

47. The process of claim 45, wherein the lubricant comprises up to about 10% of the dry weight of the taste masking coating composition.

48. The process of claim 41, wherein the coating comprises between about 10% w/w and about 40% w/w of the active pharmaceutical ingredient-containing core.

49. (canceled)

50. (canceled)

51. (canceled)

52. (canceled)

53. (canceled)

54. (canceled)

55. (canceled)

56. (canceled)

57. The process of claim 41, wherein the drug comprises one or more of alkaloids, antacids, analgesics, anabolic agents, anti-anginal drugs, anti-allergy agents, anti-arrhythmia agents, antiasthmatics, antibiotics, anticholesterolemics, anticonvulsants, anticoagulants, antidepressants, antidiarrheal preparations, anti-emetics, antihistamines, antihypertensives, anti-infectives, anti-inflammatories, antilipid agents, antimanics, anti-migraine agents, antinauseants, antipsychotics, antistroke agents, antithyroid preparations, anabolic drugs, antiobesity agents, antiparasitics, antipsychotics, antipyretics, antispasmodics, antithrombotics, antitumor agents, antitussives, antiulcer agents, anti-uricemic agents, anxiolytic agents, appetite stimulants, appetite suppressants, beta-blocking agents, bronchodilators, cardiovascular agents, cerebral dilators, chelating agents, cholecystekinin antagonists, chemotherapeutic agents, cholesterol reducing agents, cognition activators, contraceptives, coronary dilators, cough suppressants, CNS drugs, decongestants, diabetes agents, diuretics, emollients, enzymes, erythropoietic drugs, expectorants, fertility agents, fungicides, gastrointestinal agents, growth regulators, hormone replacement agents, hyperglycemic agents, hypoglycemic agents, ion-exchange resins, laxatives, migraine treatments, mineral supplements, mucolytics, narcotics, neuroleptics, neuromuscular drugs, non-steroidal anti-inflammatories (NSAIDs), nutritional additives, peripheral vasodilators, polypeptides, prostaglandins, psychotropics, renin inhibitors, respiratory stimulants, sedatives, steroids, stimulants, sympatholytics, thyroid preparations, tranquilizers, uterine relaxants, vaginal preparations, vasoconstrictors, vasodilators, vertigo agents, vitamins, wound healing agents, and others.

58. The process of claim 41, further comprising formulating the taste-masked pharmaceutical composition as sprinkles, a dry powder, a suspension, an emulsion, whole chewable tablets, or dispersible tablets.

59. The process of claim 41, wherein the taste-masking coating composition is applied to the drug.

60. The process of claim 41, wherein the taste masking coating composition further comprises one or more of a plasticizer, a coloring agent, and a gloss producer.

61. A process for preparing a taste-masked pharmaceutical composition, the process comprising:
coating one or more microcrystalline cellulose beads with a suspension containing at least one active pharmaceutical ingredient to form one or more drug loaded beads;
coating the drug loaded beads with a taste masking coating composition comprising (i) 25% w/w of the total taste masking coating composition of a copolymer of acrylate and methacrylate with a quaternary ammonium group in combination with sodium carboxymethylcellulose and (ii) 68.5% w/w of the total taste masking coating composition of polyvinyl alcohol-polyethylene glycol copolymer.

62. A method of treating, preventing or diagnosing a disease condition by orally administering a taste-masked pharmaceutical composition to a patient in need thereof, the pharmaceutical composition comprising a core containing an active pharmaceutical ingredient and a taste masking coating composition, the taste masking coating composition forming a coat around at least a portion of the core and comprising a combination of (i) a copolymer of acrylate and methacrylate with a quaternary ammonium group in combination with sodium carboxymethylcellulose and (ii) a polyvinyl alcohol-polyethylene glycol copolymer.
